(19) United States
(12) Patent Application Publication
(10) Pub. No.: US 2021/0327559 A1
(43) Pub. Date: Oct. 21, 2021

(54) System for Optimizing Behavioral Changes of a User to Improve the User's Wellbeing

(71) Applicant: Koa Health B.V., Barcelona (ES)

(72) Inventors: Oliver Harrison, London (GB); Aleksandar Matic, Lloret de Mar (ES)

(21) Appl. No.: 17/359,620

(22) Filed: Jun. 27, 2021

Related U.S. Application Data
(63) Continuation-in-part of application No. PCT/EP2019/087039, filed on Dec. 26, 2019.

(30) Foreign Application Priority Data
Dec. 27, 2018 (EP) 18382996.9
Dec. 26, 2019 (EP) PCT/EP2019/087039

(51) Int. Cl. G16H 20/70; G16H 50/20; G16H 40/67; A61B 5/16; G06F 16/2457

(52) U.S. Cl. CPC G16H 20/70; G16H 50/20; G06F 16/24575; A61B 5/165; G16H 40/67

(57) ABSTRACT

A method for optimizing a user's behavioral changes uses an artificial intelligence system to present actions to the user for achieving a behavioral change goal. The goal is first set at a time instant t. A user characteristics vector and current state vector are generated using static and dynamic user characteristics describing the user's current state. The vectors are used to generate a behavioral change tool vector for the time instant t. The behavioral change tool vector is presented to the user to select a suggested action for achieving the goal. The user characteristics, current state, and behavioral change tool vectors for the time instant t are mapped to a next current state vector for a next time instant t+1. The system repeats generating the behavioral change tool vector using vectors updated for the next time instant t+1 until the mapping indicates that the behavioral change goal has been achieved.

### CROSS REFERENCE TO RELATED APPLICATION

This application is filed under 35 U.S.C. §111(a) and is based on and hereby claims priority under 35 U.S.C. §120 and §365(c) from International Application No. PCT/EP2019/087039, filed on Dec. 26, 2019, and published as WO 2020/136217 A1 on Jul. 2, 2020, which in turn claims priority from European Application No. EP18382996.9, filed in the European Patent Office on Dec. 27, 2018. This application is a continuation-in-part of International Application No. PCT/EP2019/087039, which is a continuation of European Application No. EP18382996.9. International Application No. PCT/EP2019/087039 is pending as of the filing date of this application, and the United States is an elected state in International Application No. PCT/EP2019/087039. This application claims the benefit under 35 U.S.C. §119 from European Application No. EP18382996.9. The disclosure of each of the foregoing documents is incorporated herein by reference.

### TECHNICAL FIELD

The present invention has its application within the telecommunications sector and relates to the deployment of tools in telecommunications network entities (e.g., applications in mobile user terminals such as smartphones, tablets, wearable programmable electronic devices, computer programs, etc.) that work systematically with a user to improve the user's personal motivation for wellbeing. More particularly, the present invention involves a system and method for optimizing behavioral changes of an individual.

### BACKGROUND

Since 2008, the leading causes of death in the United States have resulted from personal choices, and the same trend is seen in many countries worldwide. In this context, behavior change programs that address these personal choices have the potential to deliver considerable benefits to both the general health of the population and to the cost burden of disease across the population. For many people, changing health-related behavior is very challenging. For this reason, despite decades of health education, smoking rates have plateaued, and obesity rates are rising worldwide.

To achieve a positive impact on health and well-being, behavior change services have deployed a range of approaches that have typically required an extended deployment period.

U.S. patent publication 2010/153287 discloses a system and method that guides patients, caregivers and supporters of individuals as they confront, and endeavor to overcome, a serious health crisis or major life transition. The system facilitates the reduction of the overwhelming psychological nature of the diagnosis event by compartmentalizing it into manageable phases, based on distance from diagnosis, enabling the patient to overcome and quickly envision the patient's first step toward wellbeing.

U.S. patent publication 2018/068573 discloses a method for influencing a user's perception of a subject. The method obtains a desired value associated with a sensory trigger that is relevant to the user's perception and is configured to target an automatic mind of the user. Via a feedback interface, a subconscious response is stimulated to influence the user's perception.

U.S. patent publication 2014/323817 discloses a computer-implemented method for mental state analysis by correlating mental state information of an individual with mental state information from a plurality of people and categorizes the individual with the others based on the correlation.

International application publication WO2017/176653 discloses the generation of predictive models to suggest beneficial actions based on a user's data over time. The system delivers the suggested action or circumstance to a device carried by or worn by a user in the form of advice or a story, using text, speech, images, or video.

U.S. patent publication 2010/037170 discloses a system configured to provide to a graphical user interface enabling a user to establish and monitor progress towards a personal objective, and to receive data input from the user.

One reason for the limited impact of behavior change programs to date is that advice from healthcare professionals and public health campaigns is not personalized for each user. In addition, for people embarking on behavior change programs, the extent of the change (e.g., moving from being a 60-cigarette-per-day smoker to becoming a non-smoker, or losing 20% of one's body weight) often creates an overwhelming sense of challenge, and thus a significant psychological barrier to both getting started and making positive progress.

There are a myriad of behavioral change theories. It is, however, unclear how to personalize the behavioral change approach to an individual for improved effectiveness. Therefore, there is a need in the state of the art for developing a system that identifies an optimal path based on the personalized selection of behavior change approaches to optimize the effectiveness of the behavior change.

### SUMMARY

A system and method for optimizing the desired behavioral changes of a user involves an artificial intelligence system that outputs a behavioral change tool vector, which presents suggested actions to the user to achieve the user's desired behavioral change goal. For a time instant t, the system acquires static user characteristics in order to generate a user characteristics vector. For the time instant t, the system acquires dynamic user characteristics that define a current user state in order to generate a user current state vector. The artificial intelligence system is queried using the generated vectors as input to generate as output a behavioral change tool vector for the time instant t. The behavioral change tool vector is delivered to the user, who makes selections of suggested actions to change the user's behavior based on the output of the artificial intelligence system. The system maps the vectors that are generated for the time instant t to a next current state vector for a next time instant t+1. The querying of the artificial intelligence system is repeated using as input the three vectors updated over time for the next time instant t+1 until the user's behavioral change goal is achieved. The selections made by the user define an optimal path for achieving the user's desired behavioral changes.

A method for optimizing the desired behavioral changes of a user uses an artificial intelligence system to generate and present a suggested action to the user for achieving the user's behavioral change goal. The behavioral change goal is first set at a time instant t. Static user characteristics are acquired, and a user characteristics vector is generated using the static user characteristics. Dynamic user characteristics are acquired that describe a current user state of the user, and a current state vector is generated using the dynamic user characteristics. The artificial intelligence system is queried using as input the user characteristics vector and the current state vector to generate as output a behavioral change tool vector for the time instant t. The behavioral change tool vector is presented to the user for the user to select the suggested action for achieving the behavioral change goal. The behavioral change tool vector includes a set of actions from which the user selects a suggested action. The user characteristics vector, the current state vector, and the behavioral change tool vector generated for the time instant t are mapped to a next current state vector generated for a next time instant t+1. The querying to the artificial intelligence system is repeated using as input the user characteristics vector, the current state vector, and the behavioral change tool vector, each updated for the next time instant t+1, until the mapping indicates that the behavioral change goal has been achieved.

In one embodiment, the current state vector is generated using the dynamic user characteristics that are acquired by using an ambient sensor on a personal communications device of the user. In another embodiment, the current state vector is generated using the dynamic user characteristics that are acquired by direct manual input from the user onto the personal communications device of the user.

In one embodiment, the user characteristics vector is generated using the static user characteristics that are acquired by using the ambient sensor on the personal communications device of the user. In another embodiment, the static user characteristics are acquired by direct manual input from the user onto the personal communications device of the user. In yet another embodiment, the static user characteristics are acquired from an external database.

An electronic system for optimizing behavioral changes of a user includes a processor, a database and a personal communications device of the user. Static user characteristics and dynamic user characteristics are stored in the database. The dynamic user characteristics describe a current user state of the user. The processor implements an artificial intelligence model that is built on the database. The processor is configured to generate and map vectors and to present a suggested action to the user for achieving a behavioral change goal of the user. The processor is configured to generate a user characteristics vector using the static user characteristics. A current state vector is generated using the dynamic user characteristics that are acquired by using an ambient sensor on the personal communications device of the user. Alternatively, the dynamic user characteristics are acquired by direct manual input from the user onto the personal communications device of the user.

A behavioral change tool vector for a time instant t is generated using the user characteristics vector and the current state vector by implementing the artificial intelligence model. The artificial intelligence model generates the behavioral change tool vector using reinforcement learning. The behavioral change tool vector is presented to the user for the user to select the suggested action for achieving the user's behavioral change goal. The behavioral change tool vector includes a set of actions from which the user selects the suggested action. The processor is configured to map the user characteristics vector, the current state vector, and the behavioral change tool vector generated for the time instant t to a next current state vector generated for a next time instant t+1. The generating of the behavioral change tool vector is repeated using as input the user characteristics vector, the current state vector, and the behavioral change tool vector updated for the next time instant t+1 until the mapping to the next current state vector indicates that the behavioral change goal has been achieved.

Other embodiments and advantages are described in the detailed description below. This summary does not purport to define the invention. The invention is defined by the claims.

### BRIEF DESCRIPTION OF THE DRAWING

The accompanying drawings, where like numerals indicate like components, illustrate embodiments of the invention.

FIG. 1 shows a flow diagram of a method for optimizing the behavioral changes of a user.

FIG. 2 shows a schematic block diagram of a system for acquiring user state and user characteristics to be used in defining an optimal path for the user's behavioral changes.

FIG. 3 shows an artificial intelligence system for determining the steps of the optimal path for the behavioral changes based on the acquired user state and user characteristics.

### DETAILED DESCRIPTION

Reference will now be made in detail to some embodiments of the invention, examples of which are illustrated in the accompanying drawings.

A novel system identifies an optimal path for an individual user at a specific time to change the user's behavior based on a model that combines user characteristics, user preferences, and data from other users. The invention provides a tool to personalize the selection of behavior change approaches and to compartmentalize the overall behavior change program into a personalized set of more manageable steps. Both the approach selection and the concatenation of steps are derived from a mathematical model that combines user characteristics, user preferences, and data from other users. The data from other users is matched for relevance to the system's user. Thus, the identified optimal path includes both the defined steps in which specific approaches are deployed, and the sequence of those steps over time. The aim is to enable the user quickly to envision simple first steps toward well-being and to maintain personal motivation across each step through the positive psychological impact of successfully completing interim steps.

The novel method follows an “optimal path” in the selection of approaches and in the concatenation of steps defined for each user. The optimal path of the method passes through a vector space that includes potential behavior change approaches implemented over time. The sequence of steps constitutes a behavioral change program at an individual level.

The vector space includes current and target user behavior, potential behavior change approaches and sequencing of individual steps over time. The optimal path is determined by dynamically setting each step in the behavioral change program through an artificial intelligence system built on a database containing the vector space. The artificial intelligence system is trained by machine learning. The input to the artificial intelligence system includes the parameters: user characteristics, user states, and information about the administered behavioral change tool over time.

In order to select the steps that define an optimal path, critical user characteristics are identified, including: semi-static or static characteristics (e.g., risk appetite, time discounting, biological gender, Big-5 personality); and dynamic characteristics (e.g., mood, momentary happiness, boredom states).

The system identifies the critical user characteristics using tools derived from Personal Construct Theory (e.g., manual/automated repertory grids) that enable the quantitative estimation of positive and negative associations. In addition, the system analyzes empirical data from previous trials of other users in which behavior change approaches were matched to users, and the effectiveness of those approaches was quantified. Furthermore, continuous adaptation of the aforementioned analysis is carried out based on reinforcement learning or other existing approaches.

Finally, the optimal sequencing of individual approaches for each step is mapped into an overall behavior change program, which is called concatenation.

In one embodiment, a computer-implemented method for optimizing behavioral changes of a user generates a behavioral change goal set for the user. Static user characteristics are acquired relating to a specific time instant, t, and are used to generate a user-characteristics vector and dynamic user characteristics. The user-characteristics vector and the dynamic user characteristics are then used to generate a user current state vector. The user current state vectors and the generated user characteristics are then input into an artificial intelligence system. A behavioral change tool vector for the specific time instant, t, is generates using artificial intelligence.

The behavioral change tool vector is delivered as the output to the user, who makes one or more selections designed to change the user's behavior based on the behavioral change tool vector. The method maps the user characteristics vector, the user current state vector and the behavioral change tool vector generated for the specific time instant, t, to a next current state vector generated for a next time instant, t+1. The query to the artificial intelligence system is then repeated using as the input the three vectors updated over time until the mapping indicates that the behavioral change goal has been achieved. The selections made by the user define the optimal path for achieving behavioral changes.

A another embodiment, a system is built on a database and configured to implement the method described above using artificial intelligence. The database and an artificial intelligence processing means form an artificial intelligence system. The artificial intelligence system generates the behavioral change tool vector as an output to the user. The artificial intelligence system uses as its input the generated user characteristics vector and the user current state vector stored in the database and updated over time.

The method and system of the embodiments described above have a number of advantages over the prior art, including the following:

Greater effectiveness: The novel method reduces the psychological barrier to embarking upon, and achieving, behavioral change by compartmentalizing the overall vector into steps over time, where each step is selected based on the likelihood of both being effective and engaging the user at the specific time. At each step, the method identifies the most effective approach at that time, and identifies a sequence of subsequent steps that will maximize the effectiveness over time. By tailoring approaches and tools to individual user characteristics, optimal paths increase user uptake, adherence to a health and well-being improvement program, the efficacy of individual approaches and tools, and the improvement in overall health and well-being.

Continuous improvement: Optimal path calculations are continuously updated based on empirical evidence of the effectiveness of various approaches, tools and programs. Through a reinforcement learning mechanism, optimal paths are refined to maximize their effectiveness. Such learnings are incorporated into the optimal path service on a real-time basis and are available to subsequent users.

Cost savings: Because optimal path calculations are automated, they represent a lower-cost approach to delivering the functionality than human or manual approaches that exist in the prior art.

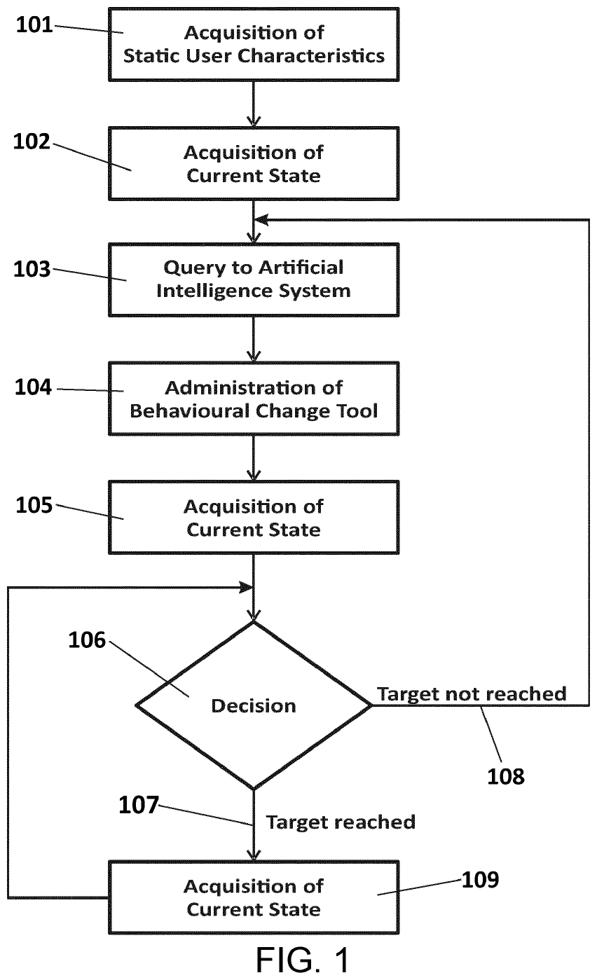

FIG. 1 shows a flow diagram of a novel method for optimizing the user's behavioral changes that is implemented using a novel system. Given a behavioral change goal, the method starts with the acquisition of a set of personal static user characteristics (step 101). In step 102, the system acquires the current state of the user. In step 103, the personal static user characteristics and the user's current state are fed as inputs into an artificial intelligence system 400 as a query. In step 104, the artificial intelligence system 400 outputs the recommended behavioral change tool with its features. In step 105, the user's current state is updated the decision is made (step 106) regarding whether the user's behavioral change goal has been reached. If the behavioral change goal has not been achieved (step 108), a new query to the artificial intelligence system 400 is made (step 103) in order to obtain an adjusted behavioral change tool or program. If the behavioral change goal has been reached (step 107), the user's current state is acquired (step 109) over time to ensure that the behavioral change goal is maintained.

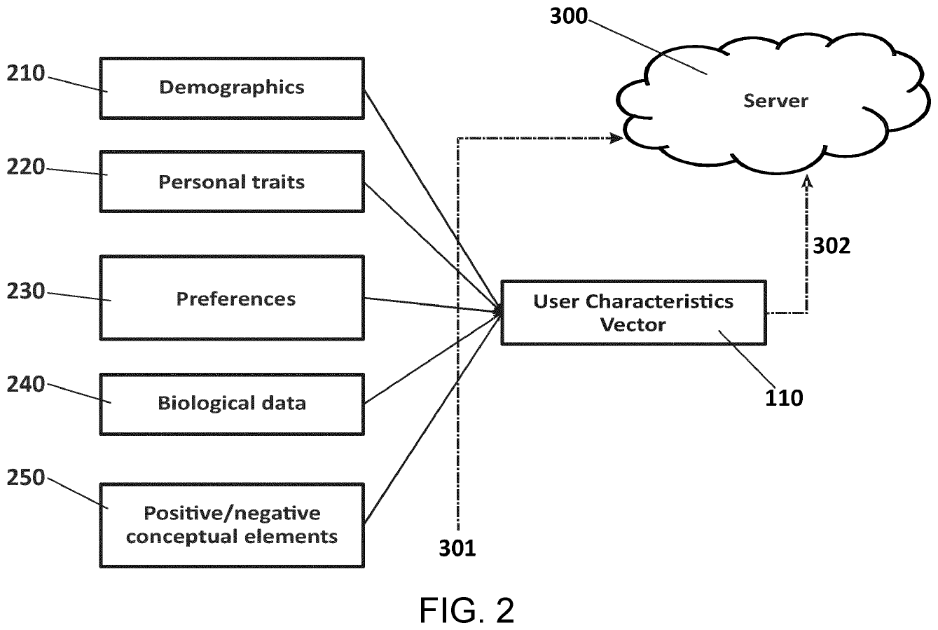

FIG. 2 is a schematic block diagram of a module of the system for acquiring user state and user characteristics to be used in defining the optimal path for the user's behavioral changes. FIG. 2 illustrates that the user characteristics vector 110 is input into the artificial intelligence system 400. The module for acquiring user state and user characteristics includes at least five sub-modules for acquiring various types of information. The demographic information module 210 acquires information relating to gender, age, ethnic group, religion, socio-economic status, relationship status, etc. of the user. The personal traits module 220 acquires information relating to the BIG-5 personality traits, boredom proneness, or even cognitive abilities such as IQ. The preferences module 230 acquires information relating to a wide range of personal descriptors, such as the inclination towards specific products, items, colors, hobbies, topics, ideas, etc. The biological characteristics module 240 acquires information relating to genetics and physiological measurements. And the positive/negative conceptual associations module 250 determines object associations in the domain space of the targeted behavioral change. For example, in the case of dietary goals, module 250 determines associations that may include links between specific food categories, drinks, feelings after eating, obesity, and well-being.

The sources of the information that is acquired by the demographics module 210, the personal traits module 220, the preferences module 230, the biological data module 240, and the positive/negative conceptual elements module 250 include direct inputs from the user, external data bases, external services, and data from sensors.

In one embodiment, all of this information is sent (step 301) to a server 300 of the novel system that generates the user characteristics vector 110 based on the received information or data. In another embodiment, the user characteristics vector 110 is computed locally at the client side (at the user's equipment) by extracting relevant features that can be anonymous or anonymized. Then the user characteristics vector 110 is sent (step 302) to the server 300.

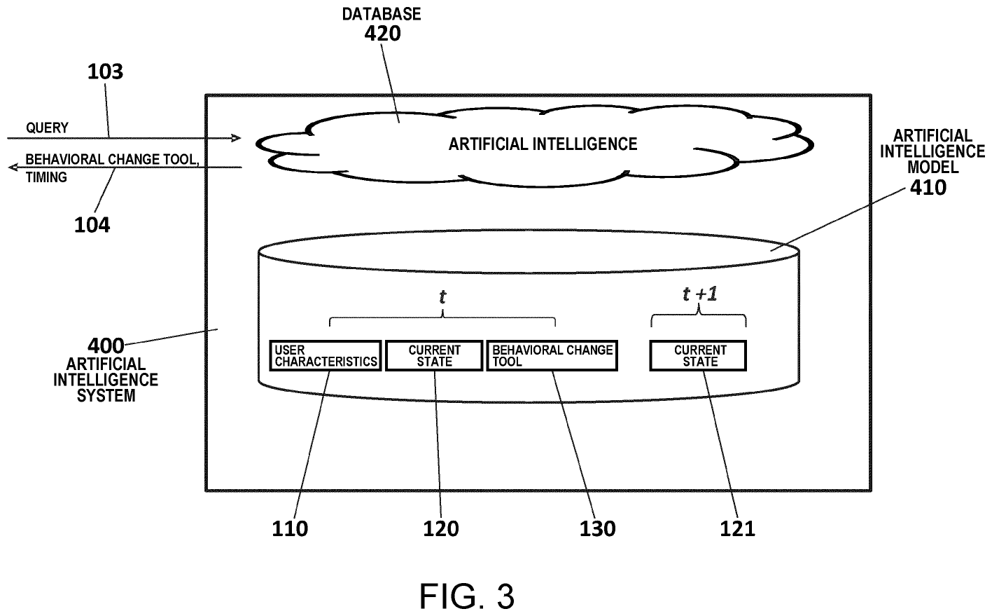

FIG. 3 shows the artificial intelligence system 400 for setting the steps of the optimal path for the behavioral changes based on the acquired user state and characteristics. FIG. 3 illustrates that the current user state vector 120 is one of the components of the artificial intelligence model 410 (also called a machine learning model). The user state vector 120 is a dynamically changing vector comprised of three sub-vectors: the current behavior vector, the current personal characteristics vector and the emotional status vector.

Current behavior vector. The sub-vector of current behaviors represents the behavioral domain of the targeted behavioral change. But the vector may also include the patterns of activities of daily living, such as sleep, diet, sports, social activities, and whereabouts.

Current (dynamic) personal characteristics vector. The sub-vector of dynamic personal characteristics relates to momentary cognitive processing, such as risk appetite, executive cognitive functions, and time discounting.

Emotional status vector. The sub-vector of emotional status concerns the assessment of the user's emotional and wellbeing indexes.

The three sub-vectors are generated using internal or external services that acquire information from personal communications devices, ambient sensors and direct user inputs. The personal communications devices include mobile phones, tablets, computers, and wearables that measure physical or physiological signals. The ambient sensors include presence sensors (motion detectors), audio/video sensors, temperature sensors, and air monitors. The direct user inputs are acquired through questionnaires, surveys, and notifications.

The behavioral change tool or program that is output by the artificial intelligence system 400 in step 104 is a behavioral change tool vector 130 of the artificial intelligence model 410, as illustrated in FIG. 3. The behavioral change tool vector 130 includes a start (trigger), content and an end. The behavioral change tool vector 130 is delivered in step 104 by the artificial intelligence system 400 to the user as an action to be performed. The types of suggested actions that the behavioral change tool vector 130 instructs the user to take include:

generic tips such as “Go to bed each day at the same time makes you rest better”;

motivational advice such as quotes from Buddha or Einstein or a thought of the day;

personal tips such as “To make use of your time during your commute, try TED Talks”;

mini goals such as “Sleep better than your mother this week”, “Take a different route now”

points of decision such as personalized prompts, rules, feedback from third-party experience at bank, supermarket, etc.;

educational actions such as using educational cards, mini surveys, videos, audio guides;

chat actions such as book a table using OpenTable, find a different route to work using Google Maps;

games such as attention training, cognitive test games, and anti-bias balloons;

exercise such as exposure exercise, rethink your thoughts;

conversations such as TLC conversations on stress, conversations through educational cards, chat actions and games;

suggesting apps such as “Why not use Strava for running. Click here”;

improvement programs such as run 20 km in 10 days, take cognitive behavioral therapy (CBT) for body dysmorphic disorder (BDD), use a mix of other tools;

notifications such as “Tell us how you are feeling” and priming messages;

use a buddy system such as “Get motivation by helping others or get help yourself”; and

consult an expert such as a therapist, a financial advisor, a gym coach, a personal trainer.

The artificial intelligence system 400 includes the artificial intelligence model 410 and is built on a database 420. The database 420 contains the user characteristics vector 110, the user current state vector 120, and the behavioral change tool vector 130. The three vectors 110, 120, 130 relate to a specific time instant t and are mapped to the next current state vector 121, which is measured at a specific next time instant t+1. In one embodiment, the artificial intelligence model 410 is based on reinforcement learning. The state space in model 410 is represented by a subset of elements in the current state vector 120 measured at time t. The context in model 410 is represented by a subset of elements in the current state vector 120 and in the personal characteristics vector 110 measured at time t. The reward in model 410 is represented by a subset of elements in the next current state vector 121 measured at t+1.

Another embodiment defines the optimal path as a component of a well-being improvement service. The calculation of optimal paths for individual users to achieve health and well-being goals is primarily conceived as a component of a well-being improvement service. As such, optimal path calculations tailor the specific behavior change approaches and tools provided for an individual user over time. The behavior change approaches and tools are tailored based on the user's personal characteristics and the overall behavior change goal that the user has set. Another embodiment determines the optimal path as a service for third parties. In addition, the calculation of optimal paths for individual users to achieve health and well-being goals may be offered as a service for third-party health and wellbeing improvement systems. Such an optimal path as a service requires defined inputs from the third party, including personal characteristics of the service user, the user's health and wellbeing goal, and the range of approaches and tools available to help the user to achieve his goal.

Although the present invention has been described in connection with certain specific embodiments for instructional purposes, the present invention is not limited thereto. The embodiments of the invention can be implemented in a variety of architectural platforms, operating and server systems, devices, systems, or applications. Accordingly, various modifications, adaptations, and combinations of various features of the described embodiments can be practiced without departing from the scope of the invention as set forth in the claims.

1-24 (canceled)

25. A method for optimizing behavioral changes of a user, comprising:
setting a behavioral change goal for the user for a time instant t;
acquiring static user characteristics;
generating a user characteristics vector using the static user characteristics;
acquiring dynamic user characteristics that describe a current user state;
generating a current state vector using the dynamic user characteristics;
querying an artificial intelligence system using as input the user characteristics vector and the current state vector to generate as output a behavioral change tool vector for the time instant t;
presenting the behavioral change tool vector to the user for the user to select a suggested action for achieving the behavioral change goal;
mapping the user characteristics vector, the current state vector, and the behavioral change tool vector generated for the time instant t to a next current state vector generated for a next time instant t+1; and
repeating the querying to the artificial intelligence system using as input the user characteristics vector, the current state vector, and the behavioral change tool vector, updated for the next time instant t+1, until the mapping indicates that the behavioral change goal has been achieved.

26. The method of claim 25, wherein the behavioral change tool vector includes a set of actions from which the user selects the suggested action.

27. The method of claim 25, wherein the current state vector includes data related to patterns of activities of daily living of the user, data related to momentary cognitive processing of the user, and data related to the user's emotional status.

28. The method of claim 25, wherein the current state vector is generated using the dynamic user characteristics that are acquired by using an ambient sensor on a personal communications device of the user.

29. The method of claim 25, wherein the current state vector is generated using the dynamic user characteristics that are acquired by direct manual input from the user onto a personal communications device of the user.

30. The method of claim 25, wherein the static user characteristics include information selected from the group consisting of: demographic information describing the user, personal traits of the user, personal preferences of the user, biological characteristics of the user, a positive association of the user of a first object to the behavioral change goal, and a negative association of the user of a second object to the behavioral change goal.

31. The method of claim 25, wherein the user characteristics vector is generated using the static user characteristics that are acquired by using an ambient sensor on a personal communications device of the user.

32. The method of claim 25, wherein the user characteristics vector is generated using the static user characteristics that are acquired from an external database.

33. The method of claim 25, wherein the user characteristics vector is generated using the static user characteristics that are acquired by direct manual input from the user onto a personal communications device of the user.

34. An electronic system for optimizing behavioral changes of a user, comprising:
a database in which static user characteristics and dynamic user characteristics are stored, wherein the dynamic user characteristics describe a current user state; and
a processor that implements an artificial intelligence model, wherein the artificial intelligence model is built on the database, and wherein the processor is configured to:
generate a user characteristics vector using the static user characteristics;
generate a current state vector using the dynamic user characteristics;
generate a behavioral change tool vector for a time instant t using the user characteristics vector and the current state vector by implementing the artificial intelligence model;
present the behavioral change tool vector to the user for the user to select a suggested action for achieving a behavioral change goal of the user;
map the user characteristics vector, the current state vector, and the behavioral change tool vector generated for the time instant t to a next current state vector generated for a next time instant t+1; and
repeat the generating of the behavioral change tool vector using as input the user characteristics vector, the current state vector, and the behavioral change tool vector updated for the next time instant t+1 until the mapping to the next current state vector indicates that the behavioral change goal has been achieved.

35. The electronic system of claim 34, wherein the artificial intelligence model generates the behavioral change tool vector using reinforcement learning.

36. The electronic system of claim 34, wherein the current state vector generated for the time instant t includes elements that represent a state space, wherein the current state vector and the user characteristics vector generated for the time instant t include elements that represent a context, and wherein the next current state vector generated for the next time instant t+1 includes elements that represent a reward.

37. The electronic system of claim 34, wherein the behavioral change tool vector includes a set of actions from which the user selects the suggested action.

38. The electronic system of claim 34, wherein the current state vector includes data related to patterns of activities of daily living of the user, data related to momentary cognitive processing of the user, and data related to the user's emotional status.

39. The electronic system of claim 34, further comprising:
a personal communications device of the user, wherein the current state vector is generated using the dynamic user characteristics that are acquired by using an ambient sensor on the personal communications device of the user.

40. The electronic system of claim 34, further comprising:
a personal communications device of the user, wherein the current state vector is generated using the dynamic user characteristics that are acquired by direct manual input from the user onto the personal communications device of the user.

41. The electronic system of claim 34, wherein the static user characteristics include information selected from the group consisting of:
demographic information describing the user, personal traits of the user, personal preferences of the user, biological characteristics of the user, a positive association of the user of a first object to the behavioral change goal, and a negative association of the user of a second object to the behavioral change goal.

42. The electronic system of claim 34, further comprising:
a personal communications device of the user, wherein the user characteristics vector is generated using the static user characteristics that are acquired by using an ambient sensor on the personal communications device of the user.

43. The electronic system of claim 34, wherein the user characteristics vector is generated using the static user characteristics that are first acquired from an external database.

44. The electronic system of claim 34, further comprising:
a personal communications device of the user, wherein the user characteristics vector is generated using the static user characteristics that are acquired by direct manual input from the user onto the personal communications device of the user.
